(19) United States
(12) Patent Application Publication
(10) Pub. No.: US 2012/0125774 A1
(43) Pub. Date: May 24, 2012

(54) Analysis Chip and Analysis Apparatus

(75) Inventors: Yusuke NAKAYAMA, Kyoto (JP); Koji SUGIYAMA, Kyoto (JP)

(73) Assignee: ARKRAY, Inc., Kyoto (JP)

(21) Appl. No.: 13/364,148

(22) Filed: Feb. 1, 2012

Related U.S. Application Data
(62) Division of application No. 12/515,990, filed on May 22, 2009.

(30) Foreign Application Priority Data
Apr. 27, 2007 (JP) 2007-119261

(51) Int. Cl. G01N 33/72; G01N 33/66

(52) U.S. Cl. 204/451

(57) ABSTRACT

An analysis chip that enables an apparatus to be small, analysis to be simple, analysis time to be short and analysis of both glycosylated hemoglobin and glucose to be highly accurate is provided. The electrophoresis chip includes an upper substrate 4, a lower substrate 1, a first introduction reservoir 2a, a first recovery reservoir 2b and a capillary channel for sample analysis 3x; the first introduction reservoir 2a and the first recovery reservoir 2b are formed in the lower substrate 1; and the first introduction reservoir 2a and the first recovery reservoir 2b are in communication with each other via the capillary channel for sample analysis 3x.

### TECHNICAL FIELD

The present invention relates to an analysis chip and an analysis apparatus.

### BACKGROUND ART

Analyses of both glycosylated hemoglobin and glucose are broadly performed as indicators of the condition of a living body for, e.g., the treatment or diagnosis of diabetes. Because the degree of glycosylation of hemoglobin (Hb), especially HbA1c, in blood cells reflects the history of glucose levels in a living body, it is regarded as an important indicator in the diagnosis, and treatment, or the like, in diabetes. HbA1c is HbA(α₂β₂) whose β-chain N-terminal valine has been glycosylated.

HbA1c has been analyzed by, for example, immunological methods, enzymatic methods, and high-performance liquid chromatography (HPLC) methods, among others. Although immunological methods and enzymatic methods are generally used for processing and analyzing large numbers of specimens, they are of low accuracy when determining the risk of complications. On the other hand, although HPLC methods have poorer processing capabilities than immunological methods or enzymatic methods, they are useful in determining the risk of complications. However, due to the configuration of HPLC methods, the analysis apparatus is very large and costly. On the other hand, glucose has been analyzed by, for example, enzymatic methods, and electrode methods, among others.

An example of an apparatus that can analyze both HbA1c and glucose is an apparatus that analyzes HbA1c using an immunological method and analyzes glucose using an enzymatic method. In addition, there is also an apparatus that analyzes HbA1c using an HPLC method and analyzes glucose using an electrode method. Because the latter apparatus in particular can analyze the HbA1c content of a sample (specimen) with high accuracy, it is of use in places where examinations are carried out.

However, because such conventional apparatuses are configured such that an HbA1c analyzer and a glucose analyzer are combined into a single apparatus, they are problematic due to the installation space they require, and the costs associated with the apparatus itself and the expendables required for the two analyzers. In particular, although an apparatus that takes advantage of an HPLC method analyzes HbA1c with good accuracy as described above, it has the following problems (1) to (4). (1) Due to its configuration, the analysis apparatus is very large and costly as described above. For example, there are a large number of components and it is difficult to reduce the size of a high-pressure pump, or the like. (2) It requires skill to maintain an apparatus in a condition to perform highly accurate analyses and to actually perform a highly precise analysis. (3) Large amounts of reagent are used and large amounts of liquid waste are generated. (4) Starting up the apparatus takes time even when a small number of specimens are to be analyzed. These problems apply comprehensively to the cases where both glycosylated hemoglobin, including HbA1c, and glucose are analyzed.

### DISCLOSURE OF INVENTION

Therefore, an object of the present invention is to provide an analysis chip, for the analysis of both glycosylated hemoglobin and glucose, that allows an apparatus to be small, analysis to be simple, analysis time to be short, and analysis of both glycosylated hemoglobin and glucose to be performed with high accuracy.

To achieve the object above, an analysis chip of the present invention is an analysis chip that is capable of analyzing both glycosylated hemoglobin and glucose; at least an analysis of glycosylated hemoglobin is performed by a capillary electrophoresis method;

a substrate, a plurality of fluid reservoirs and a capillary channel for the capillary electrophoresis method are included;
the plurality of fluid reservoirs includes a first introduction reservoir and a first recovery reservoir;
the capillary channel includes a capillary channel for sample analysis;
the first introduction reservoir and the first recovery reservoir are formed in the substrate; and
the first introduction reservoir and the first recovery reservoir are in communication with each other via the capillary channel for sample analysis.

An analysis apparatus of the present invention is an analysis apparatus that includes an analysis chip and an analysis unit, wherein the analysis chip is an analysis chip of the present invention.

An analysis chip of the present invention is a chip wherein a first introduction reservoir and a first recovery reservoir are formed in a substrate, and the first introduction reservoir and the first recovery reservoir are in communication with each other via a capillary channel for sample analysis. Hence, for analyses of both glycosylated hemoglobin and glucose, the present invention allows an apparatus to be small, analysis to be simple, analysis time to be short, and analysis of both glycosylated hemoglobin and glucose to be performed with high accuracy. Therefore, it is possible with an analysis chip of the present invention to accurately analyze glycosylated hemoglobin and glucose in, for example, POC (point of care) testing, and thus, to manage the risk of complications.

### BRIEF DESCRIPTION OF DRAWINGS

FIG. 1 shows diagrams illustrating an analysis chip in one working example of the present invention.

FIG. 2 is a flowchart illustrating an example of a production process of an analysis chip of the present invention.

FIG. 3 is a flowchart illustrating another example of a production process of an analysis chip of the present invention.

FIG. 4 shows diagrams illustrating an analysis chip as mentioned above that is provided with electrodes for a capillary electrophoresis method.

FIG. 5 shows diagrams illustrating an example of an analysis apparatus including an analysis chip of the present invention.

FIG. 6 shows diagrams illustrating another example of an analysis apparatus including an analysis chip of the present invention.

FIG. 7 shows diagrams illustrating an analysis chip in another working example of the present invention.

FIG. 8 shows diagrams illustrating an analysis chip in still another working example of the present invention.

FIG. 9 shows diagrams illustrating an analysis chip of still another working example of the present invention.

FIG. 10 shows diagrams illustrating an analysis chip as mentioned above that is provided with electrodes for a capillary electrophoresis method.

FIG. 11 shows diagrams illustrating still another example of an analysis apparatus including an analysis chip of the present invention.

FIG. 12 is a diagram illustrating an analysis chip of still another working example of the present invention.

FIG. 13 is a diagram illustrating an analysis chip of still another working example of the present invention.

FIG. 14 is a diagram illustrating an analysis chip of still another working example of the present invention.

### DESCRIPTION OF THE PREFERRED EMBODIMENTS

An analysis chip of the present invention may be configured such that:

the plurality of fluid reservoirs further includes a second introduction reservoir and a second recovery reservoir,
the capillary channel further includes a capillary channel for sample introduction,
the second introduction reservoir and the second recovery reservoir are formed in the substrate,
the second introduction reservoir and the second recovery reservoir are in communication with each other via the capillary channel for sample introduction,
the capillary channel for sample analysis and the capillary channel for sample introduction intersect, and
the capillary channel for sample analysis and the capillary channel for sample introduction are in communication with each other at the intersection.

An analysis chip of the present invention may be configured such that:

a first branching channel branches off from a part of the capillary channel for sample analysis,
the first branching channel is in communication with the second introduction reservoir,
a second branching channel branches off from a part of the capillary channel for sample analysis that is located on the downstream side relative to the first branching channel,
the second branching channel is in communication with the second recovery reservoir, and
the capillary channel for sample introduction is formed by the first branching channel, the second branching channel and the part of the capillary channel for sample analysis that connects the branching channels.

In an analysis chip of the present invention, the maximum length of the whole chip is in a range of, for example, 10 to 100 mm and preferably in a range of 30 to 70 mm; the maximum width of the whole chip is in a range of, for example, 10 to 60 mm; and the maximum thickness of the whole chip is in a range of, for example, 0.3 to 5 mm. The maximum length of a whole chip refers to the dimension of the longest portion of the chip in the longitudinal direction; the maximum width of a whole chip refers to the dimension of the longest portion of the chip in a direction (width direction) perpendicular to the longitudinal direction; and the maximum thickness of a whole chip refers to the dimension of the longest portion of the chip in a direction (thickness direction) perpendicular to both the longitudinal direction and the width direction.

It is preferable that an analysis chip of the present invention is such that during analyzing glycosylated hemoglobin and glucose, a diluted sample (a sample containing glycosylated hemoglobin and glucose diluted with an electrophoresis running buffer) is introduced into at least one reservoir among the plurality of fluid reservoirs, and the volume ratio of the sample: the electrophoresis running buffer is in a range of 1:4 to 1:99. The volume ratio of the sample: the electrophoresis running buffer is more preferably in a range of 1:9 to 1:59, and still more preferably in a range of 1:19 to 1:29.

In an analysis chip of the present invention, it is preferable that the capillary channel is filled with an electrophoresis running buffer.

In an analysis chip of the present invention, the maximum diameter of the capillary channel is in a range of, for example, 10 to 200 μm and preferably in a range of 25 to 100 μm; and the maximum length thereof is in a range of, for example, 0.5 to 15 cm. When the shape of the cross section of the capillary channel is not circular, the maximum diameter of the capillary channel refers to the diameter of a circle having an area that corresponds to the cross sectional area of a portion having the largest cross-sectional area.

In an analysis chip of the present invention, an inner wall of the capillary channel may be coated with a cationic group-containing compound. Examples of cationic group-containing compounds include compounds that contain cationic groups and reactive groups. Preferable examples of cationic groups include amino groups and ammonium groups. A preferable example of a cationic group-containing compound is a silylating agent that contains at least an amino group or an ammonium group. The amino group may be any of a primary, secondary or tertiary amino group.

Examples of silylating agents include: N-(2-diaminoethyl)-3-propyltrimethoxysilane, aminophenoxydimethylvinylsilane, 3-aminopropyldiisopropylethoxysilane, 3-aminopropylmethylbis(trimethylsiloxy)silane, 3-aminopropylpentamethyldisiloxane, 3-aminopropylsilanetriol, bis(p-aminophenoxy)dimethylsilane, 1,3-bis(3-aminopropyl)tetramethyldisiloxane, bis(dimethylamino)dimethylsilane, bis(dimethylamino)vinylmethylsilane, bis(2-hydroxyethyl)-3-aminopropyltriethoxysilane, 3-cyanopropyl(diisopropyl)dimethylaminosilane, (aminoethylaminomethyl)phenethyltrimethoxysilane, N-methylaminopropyltriethoxysilane, tetrakis(diethylamino)silane, tris(dimethylamino)chlorosilane, and tris(dimethylamino)silane, among others.

Among such silylating agents, those in which silicon atom(s) are substituted with titanium or zirconium may be used. Such silylating agents may be used singly or may be used in a combination of two or more.

Coating of an inner wall of a capillary channel with a silylating agent is performed, for example, as follows. First, a silylating agent is dissolved or dispersed in an organic solvent to prepare a treatment fluid. Examples of organic solvents for use in the preparation of the treatment fluid may be dichloromethane, and toluene, and the like. The concentration of the silylating agent in the treatment fluid is not particularly limited. This treatment fluid is passed through the capillary channel, and then heated. Due to this heating, the silylating agent is bonded to the inner wall of the capillary channel by covalent bonding, resulting in a cationic group being disposed on the inner wall of the capillary channel. Thereafter, washing (after-treatment) is performed with at least an organic solvent (dichloromethane, methanol, acetone, or the like), an acid solution (phosphoric acid or the like), an alkaline solution, or a surfactant solution. Although this washing is optional, it is preferable to perform such washing. Moreover, when a capillary tube that is a member independent of the substrate serves as the capillary channel, a capillary tube whose inner wall is coated with a cationic group-containing compound through the use of a commercially available silylating agent of an aforementioned kind may be used.

It is preferable that an anionic layer formed from an anionic group-containing compound is further laminated on the inner wall of a capillary channel that has been coated with a cationic group-containing compound. It is thus possible to prevent hemoglobin, or the like, present in a sample (described below) from being adsorbed onto the inner wall of a capillary channel. Moreover, due to the formation of a complex between the sample and an anionic group-containing compound and due to the electrophoresis thereof, separation efficiency is enhanced compared with electrophoresis of sample alone. As a result, analysis of glycosylated hemoglobin, or the like, can be performed more accurately in a shorter period of time. An anionic group-containing polysaccharide is preferable as the anionic group-containing compound that forms a complex with the sample. Examples of anionic group-containing polysaccharides include: sulfated polysaccharides, carboxylated polysaccharides, sulfonated polysaccharides and phosphorylated polysaccharides. Among these, sulfated polysaccharides and carboxylated polysaccharides are preferable. The sulfated polysaccharides are preferably chondroitin sulfate, and heparin, among others, with chondroitin sulfate being particularly preferable. The carboxylated polysaccharides are preferably alginic acid and salts thereof (for example, sodium alginate). There are seven types of chondroitin sulfate, i.e., chondroitin sulfate A, chondroitin sulfate B, chondroitin sulfate C, chondroitin sulfate D, chondroitin sulfate E, chondroitin sulfate H, and chondroitin sulfate K, and any of these types may be used. An anionic layer can be formed by, for example, bringing a fluid that contains an anionic group-containing compound into contact with an inner wall of a capillary channel that has been coated with a cationic group-containing compound. In this case, although a fluid for forming an anionic layer may be prepared separately, it is preferable in terms of operation efficiency that an electrophoresis running buffer that contains the anionic group-containing compound is prepared and is passed through the capillary channel whose inner wall is coated with the cationic group-containing compound.

The electrophoresis running buffer is not particularly limited, and an electrophoresis running buffer that uses an organic acid is preferable. Examples of organic acids include maleic acid, tartaric acid, succinic acid, fumaric acid, phthalic acid, malonic acid, and malic acid, among others. Preferably, the electrophoresis running buffer contains a weak base. Examples of weak bases include arginine, lysine, histidine, and tris, among others. The pH of the electrophoresis running buffer is in a range of, for example, 4.5 to 6. In the electrophoresis running buffer, the concentration of the anionic group-containing compound is in a range of, for example, 0.001 to 10 wt %.

An analysis chip of the present invention may further include a pretreatment reservoir for hemolyzing and diluting a sample containing glycosylated hemoglobin and glucose, and the pretreatment reservoir and at least one reservoir among the plurality of fluid reservoirs may be in communication with each other. It is preferable that the pretreatment reservoir be in communication with at least one of the first introduction reservoir and the second introduction reservoir, and it is more preferable that the pretreatment reservoir only be in communication with either the first introduction reservoir or the second introduction reservoir.

In the present invention, a method for analyzing glucose is not limited, and known methods can be used. A specific example is a method in which a redox reaction is carried out using glucose as a substrate and then the redox reaction is examined to analyze glucose. It is preferable in this case that an analysis chip of the present invention further contain a glucose analysis reagent, which will be described later. When an analysis chip of the present invention further includes such a glucose analysis reagent, the glucose analysis reagent may be contained in, for example, at least one reservoir among the plurality of fluid reservoirs and the pretreatment reservoir. Moreover, an analysis chip of the present invention may further include a reagent reservoir, and the glucose analysis reagent may be contained in the reagent reservoir. It is preferable in this case that the reagent reservoir is in communication with, for example, at least one reservoir among the plurality of reservoirs and the pretreatment reservoir.

Next, specific examples of glucose analysis reagents are described in combination with a method for analyzing glucose in which a reagent is applied. However, the present invention is not limited thereto.

Firstly, an example of a glucose analysis reagent is a reagent that contains a glucose oxidase, a peroxidase and a chromogenic substrate. For example, a substrate that develops a color due to oxidation is preferable as the chromogenic substrate, such as, sodium N-(carboxymethylaminocarbonyl)-4,4′-bis(dimethylamino)diphenylamine (trade name: DA-64, manufactured by Wako Pure Chemical Industries, Ltd.), 10-(carboxymethylaminocarbonyl)-3,7-bis(dimethylamino)phenothiazine or salts thereof (for example, trade name: DA-67, manufactured by Wako Pure Chemical Industries, Ltd.), hexasodium N,N,N′,N′,N″,N″-hexa(3-sulfopropyl)-4,4′,4″-triaminotriphenylmethane (for example, trade name: TPM-PS, manufactured by Dojindo Laboratories), sodium N-(carboxymethylaminocarbonyl)-4,4′-bis(dimethylamino)diphenylamine, orthophenylenediamine (OPD), and a substrate prepared by combining a Trinder's reagent and 4-aminoantipyrine, among others. Examples of Trinder's reagent include: phenol, a phenol derivative, an aniline derivative, naphthol, a naphthol derivative, naphthylamine, and a naphthylamine derivative, among others. Moreover, an aminoantipyrine derivative (i.e., vanillindiamine sulfonate, methyl benzthiazolinone hydrazone (MBTH), or sulfonated methyl benzthiazolinone hydrazone (SMBTH), among others) can be used in place of 4-aminoantipyrine. When such a glucose analysis reagent is used, glucose can be analyzed, for example, in the following manner. That is, first, a glucose oxidase is reacted with the glucose (substrate) to produce glucolactone and hydrogen peroxide. Then, due to the catalytic reaction (redox reaction) of a peroxidase that uses the thus-produced hydrogen peroxide and the chromogenic substrate as substrates, the chromogenic substrate is oxidized and develops a color. Because the extent of this color development corresponds to the amount of hydrogen peroxide, and because the amount of hydrogen peroxide corresponds to the amount of glucose, quantitative analysis of the glucose can be performed indirectly by measuring the color development.

Alternatively, a reagent that contains a redox enzyme and an electrochromic substance can also be mentioned as an example of a glucose analysis reagent. The electrochromic substance is not particularly limited insofar as, for example, the color tone thereof is changed due to the transfer of electrons. Specific examples include viologen, and viologen derivatives, among others. Examples of viologen derivatives include: diphenyl viologen, and dinitrophenyl viologen, among others. Among these, dinitrophenyl viologen is preferable. The electrochromic substances used may be commercially available, or can be prepared using known methods. Examples of redox enzymes include glucose oxidase (GOD), and glucose dehydrogenase, among others. When such a glucose analysis reagent is used, glucose can be analyzed, for example, in the following manner. That is, the glucose is reacted with the redox enzyme in the presence of an electrochromic substance. Due to this enzymatic reaction (redox reaction), electrons are liberated from the glucose. Then, due to the transfer of the liberated electrons to the electrochromic substance, the color tone of the electrochromic substance changes. Because this change in color-tone corresponds to the amount of glucose, quantitative analysis of glucose can be performed indirectly by measuring the change in color-tone.

Furthermore, a reagent that contains a redox enzyme and a tetrazolium salt having a mediator function can be mentioned as an example of a glucose analysis reagent. Examples of the redox enzyme include those that are identical to the enzymes that can be used in the reagent containing the electrochromic substance. Preferable examples of tetrazolium salts are those having at least one group from among a nitrophenyl group, a thiazolyl group and a benzothiazolyl group. Examples of tetrazolium salts include 3-(4,5-dimethyl-2-thiazolyl)-2,5-diphenyl-2H-tetrazolium bromide (MTT), 2-(4-iodophenyl)-3-(4-nitrophenyl)-5-phenyl-2H-tetrazolium chloride (INT), 3,3′-[3,3′-dimethoxy-(1,1′-biphenyl)-4,4′-diyl]-bis[2-(4-nitrophenyl)-5-phenyl-2H-tetrazolium chloride] (Nitro-TB), 2-(4-iodophenyl)-3-(4-nitrophenyl)-5-(2,4-disulfophenyl)-2H-tetrazolium monosodium salt (WST-1), 2-(4-iodophenyl)-3-(2,4-dinitrophenyl)-5-(2,4-disulfophenyl)-2H-tetrazolium monosodium salt (WST-3), 2-benzothiazolyl-3-(4-carboxy-2-methoxyphenyl)-5-[4-(2-sulfoethylcarbamoyl)phenyl]-2H-tetrazolium (WST-4), 2,2′-dibenzothiazolyl-5,5′-bis[4-di(2-sulfophenyl)carbamoylphenyl]-3,3′-(3,3′-dimethoxy-4,4′-biphenylene)ditetrazolium disodium salt (WST-5), 2-(2-methoxy-4-nitrophenyl)-3-(4-nitrophenyl)-5-(2,4-disulfophenyl)-2H-tetrazolium monosodium salt (WST-8), 2,3-bis(4-nitrophenyl)-5-phenyltetrazolium chloride, 2-(2-benzothiazolyl)-3,5-dophenyltetrazolium bromide, 2-(2-benzothiazolyl)-3-(4-nitrophenyl)-5-phenyltetrazolium bromide, 2,3-di(4-nitrophenyl)tetrazolium perchlorate, 3-(3-nitrophenyl)-5-methyl-2-phenyltetrazolium chloride, and 3-(4-nitrophenyl)-5-methyl-2-phenyltetrazolium chloride, among others. When such a glucose analysis reagent is used, glucose can be analyzed, for example, in the following manner. That is, the glucose is reacted with the redox enzyme in the presence of an aforementioned tetrazolium salt. Due to this enzymatic reaction (redox reaction), electrons are liberated from the glucose. Then, due to the transfer of the liberated electrons to the tetrazolium compound, the tetrazolium compound develops a color. Because the extent of this color development corresponds to the amount of glucose, quantitative analysis of the glucose can be performed indirectly by measuring the extent of color development.

A means of measuring the reaction between the glucose and the glucose analysis reagent is also not particularly limited, and the measurement can be carried out using a suitable optical measurement instrument. The optical measurement instrument may be a part of an analysis chip (analysis apparatus) of the present invention, or may be a separate instrument. The optical measurement instrument is not particularly limited and may be, for example, a spectrophotometer, a photosensor, a UV spectrometer, or an LED-equipped optical measurement instrument, among others. In an analysis chip (analysis apparatus) of the present invention, the components (such as an enzyme and a substrate) of a glucose analysis reagent (described above) may be disposed, for example, in a mixed state, or each component may be disposed separately and independently.

In the present invention, the method for analyzing the glucose may be, for example, an electrode method as an alternative to the method described above in which color development that occurs in association with the redox reaction is detected. In the case of an electrode method, it is preferable that an analysis chip of the present invention further includes, for example, electrodes (a cathode and an anode) for use in the electrode method and a glucose analysis reagent, and it is preferable that the electrodes and the glucose analysis reagent are disposed such that they are placed in at least one reservoir among the plurality of reservoirs and the pretreatment reservoir. In such an analysis chip, glucose can be analyzed by an electrode method, for example, using electrodes and a glucose analysis reagent. It is more preferable that electrodes used in an electrode method and the glucose analysis reagent are disposed such that they are positioned in at least one reservoir among, for example, the first introduction reservoir, the second introduction reservoir and the pretreatment reservoir. In an analysis chip of the present invention, electrodes for use in an electrode method are optional components. The electrodes for use in an electrode method may be inserted into at least one reservoir among the plurality of fluid reservoirs and the pretreatment reservoir, for example, when the analysis chip is used. The electrodes for use in an electrode method may be components of, for example, an analysis apparatus of the present invention. A specific example of a glucose analysis reagent that may be used with such an electrode method is described below. However, the present invention is not limited thereto.

An example of a glucose analysis reagent that can be used with an electrode method is a reagent that contains a redox enzyme and an electron acceptor. Examples of redox enzymes include those identical to the enzymes for use in a reagent (described above) containing an electrochromic substance. Examples of electron acceptors that may be used include: potassium ferricyanide, p-benzoquinone, phenazine methosulfate, indophenol and derivatives thereof, potassium β-naphthoquinone-4-sulfonate, methylene blue, ferrocene and derivatives thereof, osmium complexes, ruthenium complexes, NAD⁺, NADP⁺, and pyrroloquinone (PQQ), among others. When such a glucose analysis reagent is used, glucose can be analyzed, for example, in the following manner. That is, due to the catalytic reaction of the redox enzyme, glucose is oxidized simultaneously with the electron acceptor being reduced. Then, the reduced electron acceptor is reoxidized by an electrochemical technique. Because an oxidation current value obtained from this reoxidation corresponds to the amount of glucose, quantitative analysis of the glucose can be performed indirectly by measuring the current. The electrodes used in an electrode method are not particularly limited, and examples include gold electrodes, carbon electrodes, and silver electrodes, among others. The form of the electrodes used in an electrode method is also not particularly limited and, for example, they may be electrodes in which a GOD enzyme film is fixed to a film-like electrode surface (glucose electrode film).

In an analysis chip of the present invention, analysis of glucose may be carried out by, for example, a capillary electrophoresis method. The means of analysis in this case is not particularly limited, and it is preferable that, for example, an analysis chip of the present invention further include a detector that analyzes glucose by indirect absorption spectroscopy (indirect UV detection method).

Regarding analysis chips of the present invention, when analysis of glucose is carried out by a capillary electrophoresis method, it is preferable (from an analysis accuracy point of the view, and the like) that the glucose is a glucose derivative into which an ionic functional group has been introduced. The method for introducing an ionic functional group into glucose to form a derivative is not limited, and a method for forming a boric acid complex between the glucose and boric acid under alkaline conditions can be mentioned as an example. Because the boric acid complex is anionic, capillary electrophoresis is possible. A method in which a derivative of the glucose is formed with ethyl 4-aminobenzoate can be also mentioned as an example of a method for introducing an ionic functional group into glucose. Because such a derivative of glucose is cationic, capillary electrophoresis is possible.

The glycosylated hemoglobin analyzed using an analysis chip of the present invention is not particularly limited, and examples include HbA1c, labile HbA1c, and GHbLys, among others, with HbA1c being particularly preferable.

An analysis chip of the present invention may be configured such that:

a substrate includes an upper substrate and a lower substrate,
a plurality of through-holes are formed in the upper substrate,
a groove is formed in the lower substrate,
the upper substrate is laminated onto the lower substrate,
spaces created by sealing the bottom parts of the plurality of through-holes formed in the upper substrate with the lower substrate serve as a plurality of fluid reservoirs, and
a space created by sealing the upper part of the groove formed in the lower substrate with the upper substrate serves as a capillary channel.

An analysis chip of the present invention may be configured such that:

a plurality of concave portions and a groove are formed in a substrate,
a surface of the substrate is sealed with a sealing material that has openings at places corresponding to the plurality of concave portions,
the plurality of concave portions formed in the substrate serve as a plurality of fluid reservoirs, and
a space created by sealing the upper part of the groove formed in the substrate with the sealing material serves as a capillary channel.

An analysis chip of the present invention may be configured such that:

the analysis chip further includes a sealing material,
a plurality of through-holes are formed in a substrate,
a groove is formed in the bottom surface of a substrate,
the bottom surface of the substrate is sealed with the sealing material,
spaces created by sealing the bottom parts of the plurality of through-holes formed in the substrate with the sealing material serve as a plurality of fluid reservoirs; and
a space created by sealing the lower part of the groove formed in the bottom surface of the substrate with the sealing material serves as a capillary channel.

An analysis chip of the present invention may be configured such that a plurality of fluid reservoirs are in communication with each other via a capillary tube that is a member independent of the substrate, and the capillary tube may serve as a capillary channel. The material of the capillary tube is not particularly limited. Examples of the material of the capillary tube include glass, fused silica, and plastics, among others. The glass or fused silica capillary tubes used may be commercially available products. The plastic capillary tubes used may also be commercially available products, and examples include capillary tubes made from, for example, polymethylmethacrylate, polycarbonate, polystyrene, polytetrafluoroethylene (PTFE), or polyether ether ketone (PEEK), among others.

In an analysis chip of the present invention, the volumes of a plurality of fluid reservoirs are not particularly limited, and are each in a range of, for example, 1 to 1000 mm³ and preferably in a range of 50 to 100 mm³.

An analysis chip of the present invention may be configured such that the analysis chip further includes a plurality of electrodes for use with a capillary electrophoresis method, and the plurality of electrodes may be disposed such that their first ends are placed in the plurality of fluid reservoirs.

An analysis apparatus of the present invention may further include electrodes (a cathode and an anode) for use with an electrode method.

### EXAMPLES

Next, examples of the present invention are described. The present invention, however, is neither limited nor restricted by the examples below.

### Example 1

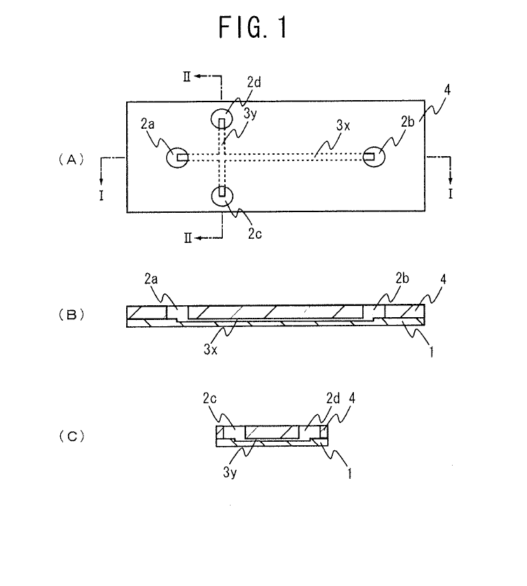

FIG. 1 shows an analysis chip of this example. FIG. 1(A) is a plan view of an analysis chip of this example, FIG. 1(B) is a cross-sectional view when taken along I-I of FIG. 1(A), and FIG. 1(C) is a cross-sectional view when taken along II-II of FIG. 1(A). For easier understanding, the size, proportions and like features of each component in the illustrations are different from the actual features of each component. This analysis chip is, as shown in the figures, configured such that an upper substrate 4 is laminated onto a lower substrate 1. A plurality of through-holes (four in this example) is formed in the upper substrate 4. The bottom parts of the four through-holes formed in the upper substrate 4 are sealed with the lower substrate 1 and, thus, fluid reservoirs 2a to 2d are formed. A cross-shaped groove is formed in the lower substrate 1. By sealing the upper part of the cross-shaped groove formed in the lower substrate 1 with the upper substrate 4, a capillary channel for sample analysis 3x and a capillary channel for sample introduction 3y are formed. The four fluid reservoirs 2a to 2d include a first introduction reservoir 2a, a first recovery reservoir 2b, a second introduction reservoir 2c and a second recovery reservoir 2d. The first introduction reservoir 2a and the first recovery reservoir 2b are in communication with each other via the capillary channel for sample analysis 3x. The first introduction reservoir 2c and the second recovery reservoir 2d are in communication with each other via the capillary channel for sample introduction 3y. The capillary channel for sample analysis 3x and the capillary channel for sample introduction 3y intersect. The capillary channel for sample analysis 3x and the capillary channel for sample introduction 3y are in communication with each other at the intersection. An analysis chip of this example is rectangular parallelepipedic. However, the present invention is not limited thereto. An analysis chip of the present invention may be in any shape insofar as it does not adversely affect the analysis of glycosylated hemoglobin and glucose. The planar shape of an analysis chip of this example is rectangular. However, the present invention is not limited thereto. The planar shape of an analysis chip of the present invention may be a square or may be of another form. In an analysis chip of this example, the maximum length of the capillary channel for sample analysis 3x and the maximum length of the capillary channel for sample introduction 3y are different. However, the present invention is not limited thereto. In an analysis chip of the present invention, the maximum length of the capillary channel for sample analysis 3x and the maximum length of the capillary channel for sample introduction 3y may be the same. Furthermore, an analysis chip of this example includes two capillary channels (3x, 3y). However, an analysis chip of the present invention is not limited thereto. For example, an analysis chip of the present invention may include the capillary channel for sample analysis 3x only. In this case, only the first introduction reservoir 2a and the first recovery reservoir 2b are formed in the lower substrate 1, and the first introduction reservoir 2a and the first recovery reservoir 2b are in communication with each other via the capillary channel for sample analysis 3x. In this analysis chip, when glucose is analyzed by an electrode method, the positions of the electrodes (a cathode and an anode) and the glucose analysis reagent (not shown) are not limited, and the electrodes and the reagent are preferably placed in at least one reservoir among the four reservoirs 2a to 2d. For example, the second introduction reservoir 2c may include therein the electrodes (a cathode and an anode) for use with an electrode method and the glucose analysis reagent. Moreover, when glucose is analyzed using, for example, a reagent that develops a color in association with a redox reaction, the site where the glucose analysis reagent is contained is not particularly limited, and it is preferable that the glucose analysis reagent is contained in, for example, at least one of the four fluid reservoirs 2a to 2d. The glucose analysis reagent may be contained in only one of the four fluid reservoirs 2a to 2d such as, for example, the second introduction reservoir 2c. Furthermore, an analysis chip of this example includes two substrate pieces (an upper substrate 4 and a lower substrate 1). However, an analysis chip of the present invention is not limited thereto. An analysis chip of the present invention may be composed of, for example, a single-piece substrate as described below.

Next, a method for producing an analysis chip of this example is described. The analysis chip, however, may be produced by methods other than the production method described below.

In an analysis chip of this example, a substrate formed from, for example, a glass material, a polymeric material or the like can be used as the lower substrate 1. Examples of the glass material include synthetic silica glass, and borosilicate glass, among others. Examples of polymeric materials include polymethylmethacrylate (PMMA), cycloolefin polymer (COP), polycarbonate (PC), polydimethylsiloxane (PDMS), polystyrene (PS), and polylactic acid, among others.

In an analysis chip of this example, the length and the width of the lower substrate 1 correspond to the maximum length and the maximum width of the whole chip as described above. Therefore, the length and the width of the lower substrate 1 are arranged to be identical to the maximum length and the maximum width of the whole chip as described above. The thickness of the lower substrate 1 in an analysis chip of this example is in a range of, for example, 0.1 to 3 mm and preferably in a range of 0.1 to 1 mm.

The material of the upper substrate 4 is not particularly limited insofar as it does not adversely affect an absorbance measurement that will be described below. For example, an upper substrate that is formed from the same material as the lower substrate 1 can be used as the upper substrate 4.

The length and the width of the upper substrate 4 are the same as the length and the width of the lower substrate 1, respectively. The thickness of the upper substrate 4 is suitably determined according to the volumes or like factors of the plurality of fluid reservoirs 2a to 2d and, for example, it is in a range of 0.1 to 3 mm and preferably in a range of 1 to 2 mm.

The width and the depth of the cross-shaped groove (the capillary channel for sample analysis 3x and the capillary channel for sample introduction 3y) are suitably determined according to the maximum diameter of the capillary channel and, for example, the width thereof is in a range of 25 to 200 μm and the depth thereof is in a range of 25 to 200 μm, and preferably the width thereof is in a range of 40 to 100 μm and the depth thereof is in a range of 25 to 200 μm. The maximum length of the capillary channel for sample analysis 3x and the maximum length of the capillary channel for sample introduction 3y are as described above.

The volumes of the plurality of fluid reservoirs 2a to 2d are as described above. In FIG. 1, the shapes of the plurality of fluid reservoirs 2a to 2d are cylindrical. However, an analysis chip of the present invention is not limited to this. In an analysis chip of the present invention, the shapes of the plurality of fluid reservoirs are not particularly limited insofar as the introduction and the recovery of a sample are not adversely affected, which will be described below and, for example, the reservoirs can be in any shape such as a quadrangular prism, a quadrangular pyramid, a cone or a combination of these shapes. Furthermore, the volumes and the shapes of the plurality of fluid reservoirs may all be the same or may each be different.

In an analysis chip of this example, the maximum thickness of the whole chip is the sum of the thickness of the lower substrate 1 and the thickness of the upper substrate 4. The maximum thickness of the whole chip is as described above.

For example, when the material of the lower substrate 1 is glass, the analysis chip can be produced as follows.

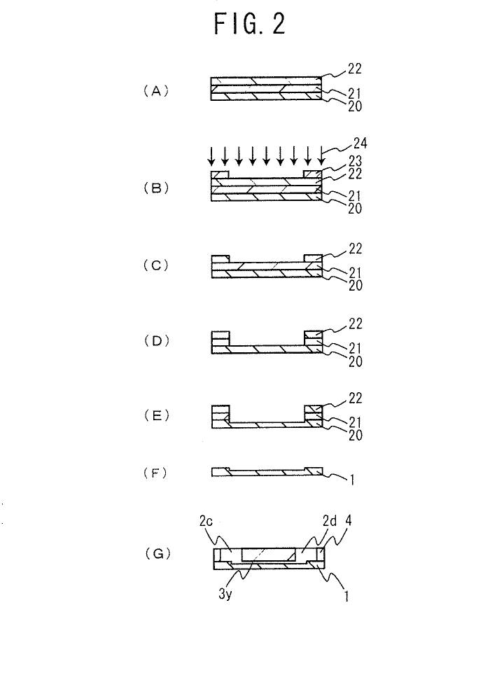

First, a surface of a glass plate 20 is masked with an alloy 21 of chromium and gold as shown in FIG. 2(A). A surface of the alloy 21 is then coated with a photoresist 22.

Next, a photosensitive film on which a layout pattern for a capillary channel for sample analysis 3x and a capillary channel for sample introduction 3y is drawn is adhered to a surface of the photoresist 22 as shown in FIG. 2(B) to prepare a photomask 23. Ultraviolet rays 24 are then irradiated over the photomask 23 for exposure.

Due to the exposure, the exposed portions of the photoresist 22 are solubilized as shown in FIG. 2(C) to form (transfer) the layout pattern on the alloy 21.

Next, the revealed portions of the alloy 21 are removed by aqua regia as shown in FIG. 2(D).

The layout pattern is then etched with hydrogen fluoride into the glass plate 20 as shown in FIG. 2(E).

Next, the photoresist 22 and the alloy 21 are removed to give the lower substrate 1 as shown in FIG. 2(F).

Next, the upper substrate 4 is prepared (not shown). A method for forming the four through-holes in the upper substrate 4 is not particularly limited. For example, when the material of the upper substrate 4 is glass, an example of a formation method is ultrasonic machining or the like. For example, when the material of the upper substrate 4 is a polymeric material, examples of a formation method include a cutting method; a molding method (such as injection molding, cast molding and press molding using a metal mold); and like methods. The four through-holes may each be formed separately or may all be formed simultaneously. When the four through-holes are formed separately, they may be formed in any order. Forming all four through-holes simultaneously by an aforementioned method that uses a metal mold or a like method requires a small number of steps and is thus preferable.

Finally, by laminating the lower substrate 1 and the upper substrate 4, an analysis chip of this example can be produced. A method for laminating the lower substrate 1 and the upper substrate 4 is not particularly limited and, and thermal welding is preferable. Although a production process was described in reference to FIG. 2, which shows cross sections corresponding to that shown in FIG. 1(C), the same production process can be applied to the cross section shown in FIG. 1(B).

For example, when the material of the lower substrate 1 is a polymeric material, the analysis chip can be produced as follows.

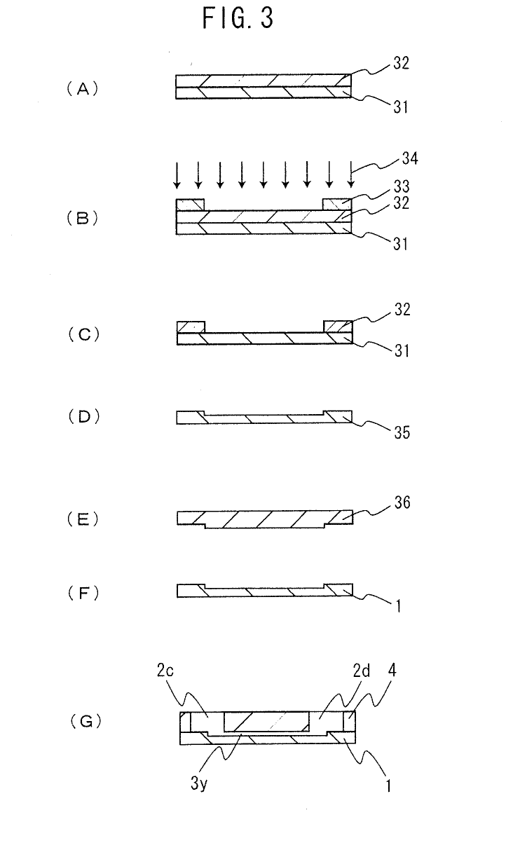

First, a surface of a silicon plate 31 is coated with a photoresist 32 as shown in FIG. 3(A).

Next, a photosensitive film on which a layout pattern for a capillary channel for sample analysis 3x and a capillary channel for sample introduction 3y is drawn is adhered to a surface of the photoresist 32 as shown in FIG. 3(B) to prepare a photomask 33. Irradiation with ultraviolet rays 34 is then performed over the photomask 33 for exposure.

Due to the exposure, the exposed portions of the photoresist 32 are solubilized as shown in FIG. 3(C) to form (transfer) the layout pattern on the silicon plate 31.

Next, the layout pattern is etched into the silicon plate 31 to prepare a base mold 35 as shown in FIG. 3(D). Examples of the etching include dry etching, and anisotropic etching, among others. The etching is preferably dry etching in view of the dimensional accuracy and the surface smoothness of the capillary channel for sample analysis 3x and the capillary channel for sample introduction 3y.

Metallic nickel electrocasting is then performed on the base mold 35 to prepare a metal mold for injection molding 36 as shown in FIG. 3(E).

Next, a lower substrate 1 composed of a polymeric material is prepared by injection molding using a metal mold for injection molding 36 as shown in FIG. 3(F).

Next, the upper substrate 4 is prepared (not shown). A method for preparing the upper substrate 4 is the same as the method used when the material of the lower substrate 1 is glass.

Finally, by laminating the lower substrate 1 and the upper substrate 4, an analysis chip of this example can be produced. A method for laminating the lower substrate 1 and the upper substrate 4 is the same as the method used when the material of the lower substrate 1 is glass. Although a production process was described in reference to FIG. 3, which shows cross sections corresponding to that shown in FIG. 1(C), the same production process can be applied to the cross section shown in FIG. 1(B).

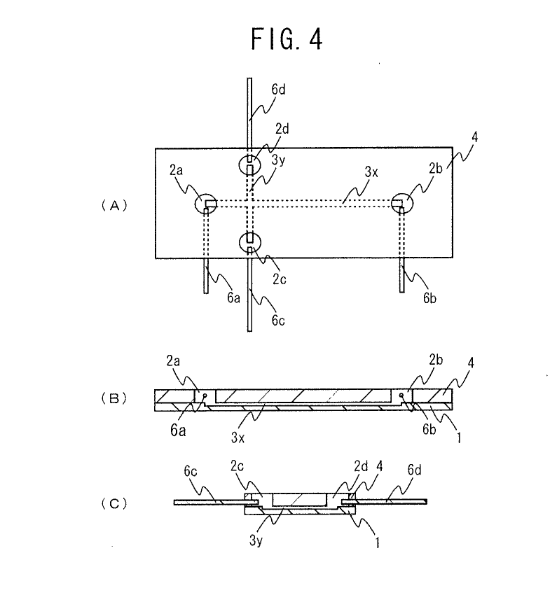

As described above, an analysis chip of the present invention may further include a plurality of electrodes for use with a capillary electrophoresis method. FIG. 4 shows an analysis chip of this example in which the plurality of electrodes for use with a capillary electrophoresis method are provided. In FIG. 4, the portions that are identical to those in FIG. 1 are given the same numbers and symbols. As shown in FIG. 4, this analysis chip has four electrodes 6a to 6d for use with a capillary electrophoresis method. The four electrodes 6a to 6d for use with a capillary electrophoresis method are disposed such that their first ends are disposed in the plurality of fluid reservoirs 2a to 2d. The four electrodes 6a to 6d for use with a capillary electrophoresis method are embedded in the upper substrate 4. The four electrodes 6a to 6d for use with a capillary electrophoresis method can be readily disposed into position by creating, in advance, introduction holes for receiving the four electrodes 6a to 6d for use with a capillary electrophoresis method in side surfaces of the upper substrate 4 when producing the upper substrate 4. In an analysis chip of the present invention, the plurality of electrodes are optional components. The plurality of electrodes may be inserted into the plurality of fluid reservoirs, for example, when the analysis chip is used.

The plurality of electrodes 6a to 6d for use with a capillary electrophoresis method may be any electrodes insofar as they are functional with an electrophoresis method. The plurality of electrodes 6a to 6d for use with a capillary electrophoresis method are each, for example, a stainless steel (SUS) electrode, a platinum (Pt) electrode, a gold (Au) electrode or the like.

An analysis chip of the present invention may further include a pretreatment reservoir for hemolyzing and diluting a sample containing glycosylated hemoglobin and glucose. A hemolysis treatment for the sample is not particularly limited and, for example, it may be a treatment in which the sample is hemolyzed with a hemolytic agent. The hemolytic agent destroys, for example, the blood cell membrane of a blood cell component present in a sample that will be described below. Examples of hemolytic agents include the aforementioned electrophoresis running buffer, saponin, and “Triton X-100” (trade name) manufactured by Nacalai Tesque, Inc., among others, with the electrophoresis running buffer being particularly preferable. It is preferable that the pretreatment reservoir be in communication with, for example, an aforementioned introduction reservoir. The pretreatment reservoir may be formed in a suitable place such as at a place near an aforementioned fluid reservoir with which the pretreatment reservoir is in communication such as, for example, the second introduction reservoir 2c. When a pretreatment reservoir is provided, a sample that will be described below is introduced into the pretreatment reservoir. The sample thus pretreated is introduced, via a channel that connects the pretreatment reservoir and an aforementioned fluid reservoir that is in communication with the pretreatment reservoir such as, for example, the second introduction reservoir 2c, into the second introduction reservoir 2c. Moreover, when there is a pretreatment reservoir and glucose is analyzed by the electrode method, for example, the pretreatment reservoir in addition to, or in place of, the at least one reservoir (for example, the second introduction reservoir 2c) of the four fluid reservoirs 2a to 2d, may contain the electrodes (a cathode and an anode) for use with the electrode method and a glucose analysis reagent. When there is a pretreatment reservoir and glucose is analyzed using a reagent that develops a color in association with a redox reaction, for example, the pretreatment reservoir in addition to, or in place of, the at least one reservoir (for example, the second introduction reservoir 2c) of the four fluid reservoirs 2a to 2d may contain the glucose analysis reagent. The pretreatment reservoir may be configured such that two reservoirs, i.e., a reservoir for hemolyzing the sample and a reservoir for diluting the sample, are in communication.

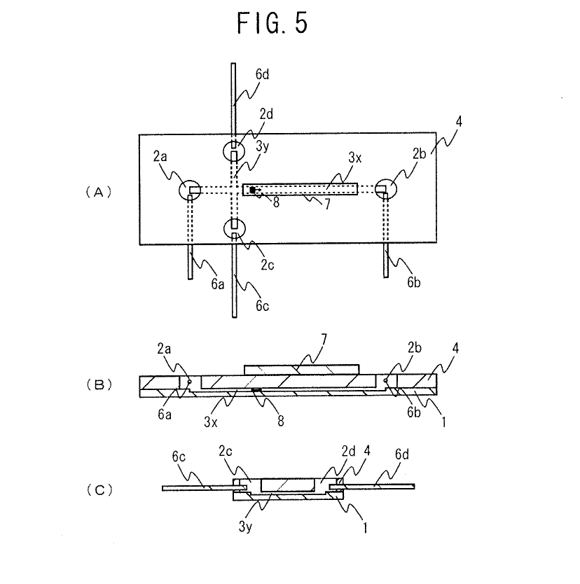

FIG. 5 shows an example of an analysis apparatus that includes an analysis chip of this example. In FIG. 5, the portions that are identical to those in FIG. 1 and FIG. 4 are given the same numbers and symbols. As shown in FIG. 5, this analysis apparatus includes an analysis unit 7. In an analysis apparatus of this example, the analysis unit 7 is a detector (line detector). The line detector is disposed on the upper substrate 4 such that the line detector is located over the capillary channel for sample analysis 3x on the first recovery reservoir 2b side relative to the intersection of the capillary channel for sample analysis 3x and the capillary channel for sample introduction 3y. A light source and a detection unit are housed in the line detector. The line detector emits light toward a sample from the light source and detects light reflected from the sample at the detection unit to measure absorbance. The analysis unit 7 is not limited to a line detector, and can be anything insofar as it can perform an analysis of glycosylated hemoglobin. The analysis unit 7 may be composed of, for example, a light source disposed under the analysis chip and a detection unit disposed in a place corresponding to where the line detector is disposed. In this case, light is emitted from the light source toward a sample, and light transmitted by the sample is detected at the detection unit to measure absorbance.

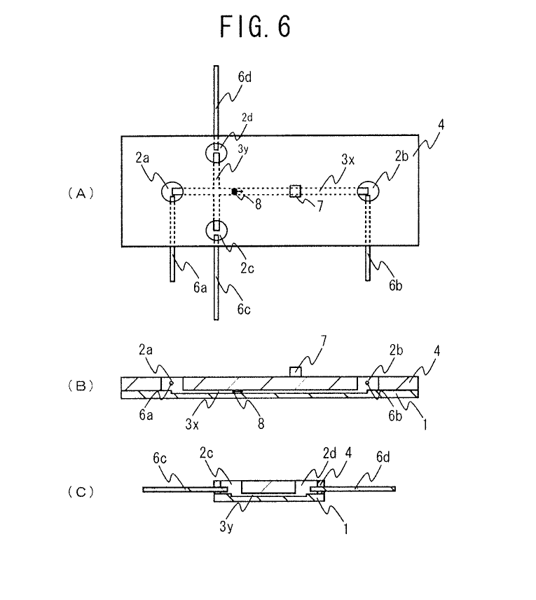

FIG. 6 shows another example of an analysis apparatus that includes an analysis chip of this example. In FIG. 6, the portions that are identical to those in FIG. 5 are given the same numbers and symbols. As shown in FIG. 6, the analysis apparatus of this example has the same configuration as the analysis apparatus shown in FIG. 5 except that the analysis unit 7 is different. As in this example, the analysis unit 7 may measure absorbance at one point.

Next, a method for analyzing glycosylated hemoglobin and glucose in connection with the present invention is described using as examples the cases where the analysis apparatus shown in FIG. 5 and FIG. 6 are used.

Analysis of glycosylated hemoglobin using an analysis apparatus (analysis chip) of this example is carried out by a capillary electrophoresis method. First, the capillary channel for sample analysis 3x and the capillary channel for sample introduction 3y are filled with an electrophoresis running buffer by pressure or capillary action. The electrophoresis running buffer is as described above.

When the capillary channels are filled with an electrophoresis running buffer in advance when the analysis apparatus is not in use (when not in analysis), it is possible to omit the step (described above) of filling with an electrophoresis running buffer and to advance immediately to the following steps, and it is thus preferable.

Next, a sample to be analyzed (a sample containing glycosylated hemoglobin and glucose) is introduced into the second introduction reservoir 2c. At this time, it is preferable to introduce a diluted sample that is diluted so as to have a volume ratio of the sample: the electrophoresis running buffer in a range of 1:4 to 1:99. That is, it is preferable that, in a method for analyzing glycosylated hemoglobin and glucose using an analysis chip (analysis apparatus) of the present invention, a diluted sample (prepared by diluting a sample containing glycosylated hemoglobin and glucose with an electrophoresis running buffer) is introduced into at least one reservoir among the plurality of fluid reservoirs, and the volume ratio of the sample the electrophoresis running buffer is in a range of 1:4 to 1:99. However, the volume ratio is not limited to this. When an analysis apparatus (analysis chip) includes a pretreatment reservoir (not shown), a sample is introduced into the pretreatment reservoir and is pretreated therein. Next, a voltage is applied to the electrode for a capillary electrophoresis method 6c and the electrode for a capillary electrophoresis method 6d to generate a potential difference between both ends of the capillary channel for sample introduction 3y, thereby moving the sample to the intersection of the capillary channel for sample analysis 3x and the capillary channel for sample introduction 3y. Examples of a sample include whole blood, hemolyzed samples prepared by subjecting whole blood to a hemolysis treatment, centrifuged blood, spontaneously precipitated blood and like samples. Examples of hemolysis treatments include sonication treatments, freeze/thaw treatments, pressure treatments, osmotic pressure treatments, and surfactant treatments, among others. The hemolysis treatment may be performed in, for example, the pretreatment reservoir. Alternatively, a sample that has been subjected to a hemolysis treatment in advance in a separate apparatus or the like may be introduced into an analysis apparatus (analysis chip). The sample may be suitably diluted with, for example, water, physiological saline, or an electrophoresis running buffer, among others. This dilution may be performed in, for example, a pretreatment reservoir. Moreover, a sample that has been subjected to a dilution treatment in advance in a separate apparatus or the like may be introduced into the analysis apparatus (analysis chip).

The potential difference between the electrode for a capillary electrophoresis method 6c and the electrode for a capillary electrophoresis method 6d is in a range of, for example, 0.5 to 5 kV.

Next, a voltage is applied to the electrode for a capillary electrophoresis method 6a and the electrode for a capillary electrophoresis method 6b to generate a potential difference between both ends of the capillary channel for sample analysis 3x. In this manner, by instantly shifting a capillary channel having different potentials at both ends from the capillary channel for sample introduction 3y to the capillary channel for sample analysis 3x, the sample 8 is moved toward the first recovery reservoir 2b side from the intersection of the capillary channel for sample analysis 3x and the capillary channel for sample introduction 3y as indicated by the arrows in FIG. 5 and FIG. 6.

The potential difference between the electrode for a capillary electrophoresis method 6a and the electrode for a capillary electrophoresis method 6b is in a range of, for example, 0.5 to 5 kV.

Next, each component of a sample that is separated due to the differences in migration speed is detected with a detector 7. It is thus possible to analyze (separate and measure) each component of a sample. According to the present invention, it is possible to analyze (separate and measure) glycosylated hemoglobin and other components of a sample that contains hemoglobin (Hb) with high accuracy.

When an analysis apparatus (analysis chip) of this example analyzes glucose by, for example, an electrode method described above, the analysis of glucose is carried out using, for example, a measuring instrument (not shown) as follows. The measuring instrument includes a power source and an ammeter. First, electrodes (a cathode and an anode) for use with an electrode method are connected to the power source, and an ammeter is disposed between a power source and the electrodes. Next, a voltage is applied to the electrodes. Thereafter, an oxidation current value is measured when a sample reaches a reservoir in which the electrodes and the glucose analysis reagent are disposed. Finally, quantitative analysis of the glucose is performed based on the oxidation current value. The measuring instrument may be a part of an analysis apparatus (analysis chip) of the present invention or may be a separate instrument.

When an analysis apparatus (analysis chip) of this example analyzes glucose by, for example, a method that uses the reagent (described above) that develops a color in association with a redox reaction, the analysis of glucose is carried out with, for example, a means that uses the optical measurement instrument described above. Specifically, the color development (change of color tone) of the glucose analysis reagent is measured when a sample reaches a reservoir in which the reagent is disposed, and quantitative analysis of the glucose is performed based on the extent of color development (change of color tone).

An analysis apparatus (analysis chip) of the present invention can analyze both glycosylated hemoglobin and glucose, and it may also be used to analyze either glycosylated hemoglobin only or glucose only. For example, first, the glucose may be analyzed, and whether or not to carry out an analysis of glycosylated hemoglobin may be determined based on the amount of glucose and other factors measured. In this manner, the diagnosis of diabetic complications and the like can be carried out more efficiently. Determination of whether or not to carry out an analysis of glycosylated hemoglobin may also be made in reference to, for example, a flow chart for diabetes diagnosis (classification of disease type). Such determination may be made automatically using, for example, a computer that is connected externally. Moreover, in this case, the type of diabetes, as classified by the computer, may be output simultaneously with the result of the glucose analysis.

Moreover, it is also possible to simultaneously analyze glycosylated hemoglobin and glucose by a capillary electrophoresis method using an analysis apparatus (analysis chip) of this example. In this case, it is preferable (from an analysis accuracy point of view and the like), as described above, that the glucose is a derivative of glucose into which an ionic functional group has been introduced. The analysis of glucose in this case can be carried out in the same manner as in the analysis of glycosylated hemoglobin using a capillary electrophoresis method described above.

### Example 2

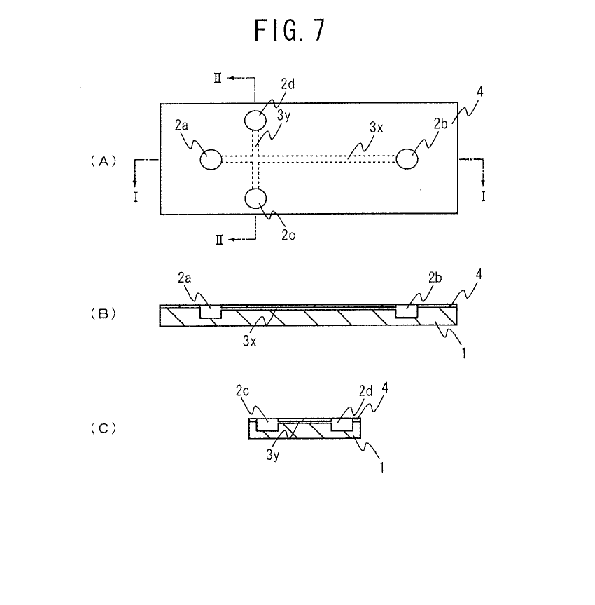

FIG. 7 shows an analysis chip of this example. In FIG. 7, the portions that are identical to those in FIG. 1 are given the same numbers and symbols. In an analysis chip of this example, a plurality of concave portions (four in this example) and a cross-shaped groove are formed in a substrate (lower substrate) 1. A surface of the substrate (lower substrate) 1 is sealed with a sealing material (upper substrate) 4 that has openings at places corresponding to the four concave portions. The four concave portions formed in the substrate (lower substrate) 1 serve as four fluid reservoirs 2a to 2d. By sealing the upper part of the cross-shaped groove formed in the substrate (lower substrate) 1 with the sealing material (upper substrate) 4, a capillary channel for sample analysis 3x and a capillary channel for sample introduction 3y are formed. Otherwise, an analysis chip of this example has the same configuration as the analysis chip shown in FIG. 1.

An analysis chip of this example can be produced, for example, as follows. However, the analysis chip may be produced by methods other than the production method described below.

For example, a substrate that is formed from the same material as the lower substrate 1 of the analysis chip shown in FIG. 1 can be used as the substrate (lower substrate) 1.

In an analysis chip of this example, the length and the width of the substrate (lower substrate) 1 correspond to the maximum length and the maximum width of the whole chip as described above. Therefore, the length and the width of the substrate (lower substrate) 1 are arranged to be identical to the maximum length and the maximum width of the whole chip as described above. The thickness of the substrate (lower substrate) 1 in an analysis chip of this example is in a range of, for example, 0.1 to 3 mm and preferably in a range of 1 to 2 mm.

The material of the sealing material (upper substrate) 4 is also not particularly limited and, for example, a substrate that is formed from the same material as the lower substrate 1 of the analysis chip shown in FIG. 1 can be used.

The length and the width of the sealing material (upper substrate) 4 are identical to the length and the width of the lower substrate 1, respectively. The thickness of the sealing material (upper substrate) 4 is in a range of, for example, 50 to 1000 μm and preferably in a range of 100 to 300 μm.

For example, a commercially available sealing material may be used for the sealing material (upper substrate) 4 after creating holes in places corresponding to the four concave portions (the four fluid reservoirs 2a to 2d).

In an analysis chip of this example, the maximum thickness of the whole chip is the sum of the thickness of the substrate (lower substrate) 1 and the thickness of the sealing material (upper substrate) 4. The maximum thickness of the whole chip is as described above.

An example of a process for producing an analysis chip of this example is described below. However, an analysis chip may be produced by processes other than the production process described below.

First, the substrate (lower substrate) 1 is prepared. A method for forming the capillary channel for sample analysis 3x and the capillary channel for sample introduction 3y in the substrate (lower substrate) 1 is not particularly limited, and the capillary channels may be formed, for example, in the same manner as in Example 1 above. A method for forming the four fluid reservoirs 2a to 2d in the substrate (lower substrate) 1 is also not particularly limited. For example, when the material of the substrate (lower substrate) 1 is glass, an example of a formation method is ultrasonic machining, or the like. For example, when the material of the substrate (lower substrate) 1 is a polymeric material, examples of a formation method include a cutting method; a molding method (such as injection molding, cast molding and press molding using a metal mold); and like methods. The four fluid reservoirs 2a to 2d may each be formed separately or may all be formed simultaneously. When the four fluid reservoirs 2a to 2d are formed separately, they may be formed in any order. Forming all four fluid reservoirs 2a to 2d simultaneously by an aforementioned method that uses a metal mold or a like method requires a small number of steps and is thus preferable.

Next, by sealing a surface of the substrate (lower substrate) 1 with the sealing material (upper substrate) 4 in which holes are created in places corresponding to the four concave portions (the four fluid reservoirs 2a to 2d), an analysis chip of this example can be produced.

The configuration of an analysis chip of this example is not limited to that shown in FIG. 7. For example, as in FIG. 4 and other figures, a plurality of electrodes may be included, and the above-described pretreatment reservoir or the like may suitably be included. The configuration of an analysis apparatus that uses an analysis chip of this example is also not particularly limited and, for example, a detector as in the analysis apparatus of FIG. 5 or FIG. 6 may be included. Moreover, a method for analyzing glycosylated hemoglobin and glucose that uses the analysis apparatus is also not particularly limited, and can be carried out, for example, in the same manner as with the case where the analysis apparatus shown in FIG. 5 or FIG. 6 is used.

### Example 3

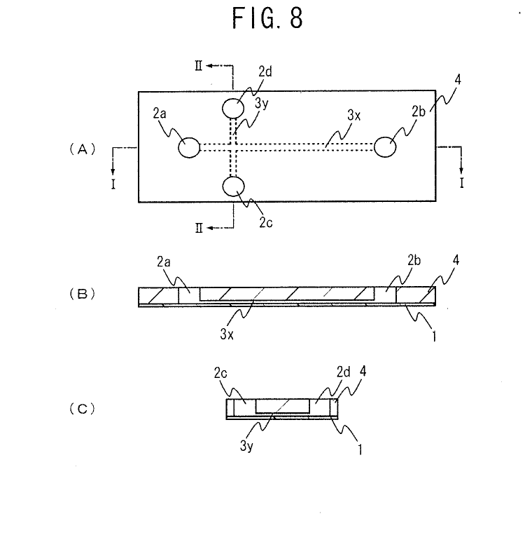

FIG. 8 shows an analysis chip of this example. In FIG. 8, the portions that are identical to those in FIG. 1 are given the same numbers and symbols. In an analysis chip of this example, a plurality of through-holes (four in this example) are formed in a substrate (upper substrate) 4. A cross-shaped groove is formed in the bottom surface of the substrate (upper substrate) 4. The bottom surface of the substrate (upper substrate) 4 is sealed with a sealing material (lower substrate) 1. The bottom parts of the four through-holes formed in the substrate (upper substrate) 4 are sealed with the sealing material (lower substrate) 1, and four fluid reservoirs 2a to 2d are formed thereby. By sealing the lower part of the cross-shaped groove formed in the substrate (upper substrate) with the sealing material, a capillary channel for sample analysis 3x and a capillary channel for sample introduction 3y are formed. Otherwise, an analysis chip of this example is of the same configuration as the analysis chip shown in FIG. 1.

An analysis chip of this example can be produced, for example, as follows. However, an analysis chip may be produced by methods other than the production method described below.

For example, a substrate that is formed from the same material as the lower substrate 1 of the analysis chip shown in FIG. 1 can be used as the substrate (upper substrate) 4.

In an analysis chip of this example, the length and the width of the substrate (upper substrate) 4 correspond to the maximum length and the maximum width of the whole chip as described above. Therefore, the length and the width of the substrate (upper substrate) 4 are arranged to be identical to the maximum length and the maximum width of the whole chip as described above. The thickness of the substrate (upper substrate) 4 in an analysis chip of this example is in a range of, for example, 0.1 to 3 mm and preferably in a range of 1 to 2 mm.

The material of the sealing material (lower substrate) 1 is also not particularly limited and, for example, a substrate that is formed from the same material as the lower substrate 1 of the analysis chip shown in FIG. 1 can be used.

The length and the width of the sealing material (lower substrate) 1 are identical to the length and the width of the substrate (upper substrate) 4, respectively. The thickness of the sealing material (upper substrate) 4 is in a range of, for example, 50 to 1000 μm and preferably in a range of 100 to 300 μm.

For example, a commercially available sealing material may be used for the sealing material (lower substrate) 1.

In an analysis chip of this example, the maximum thickness of the whole chip is the sum of the thickness of the substrate (upper substrate) 4 and the thickness of the sealing material (lower substrate) 1. The maximum thickness of the whole chip is as described above.

An example of a process for producing an analysis chip of this example is described below. However, an analysis chip may be produced by processes other than the production process described below.

First, the substrate (upper substrate) 4 is prepared. A method for forming the capillary channel for sample analysis 3x and the capillary channel for sample introduction 3y in the substrate (upper substrate) 4 is not particularly limited, and the capillary channels may be formed, for example, in the same manner as in Example 1 above. A method for forming the four through-holes in the substrate (upper substrate) 4 is also not particularly limited, and the through-holes may be formed, for example, in the same manner as in Example 1 above.

Next, by sealing the bottom surface of the substrate (upper substrate) 4 with the sealing material (lower substrate) 1, an analysis chip of this example can be produced.

The configuration of an analysis chip of this example is not limited to that shown in FIG. 8. For example, as in FIG. 4 and other figures, a plurality of electrodes for use with a capillary electrophoresis method may be included, and a pretreatment reservoir that will be described below and the like may suitably be included. The configuration of an analysis apparatus that uses an analysis chip of this example is also not particularly limited and, for example, a detector as in the analysis apparatus of FIG. 5 or FIG. 6 may be included. Moreover, a method for analyzing glycosylated hemoglobin that uses the analysis apparatus is also not particularly limited, and can be carried out, for example, in the same manner as with the case where the analysis apparatus shown in FIG. 5 or FIG. 6 is used.

### Example 4

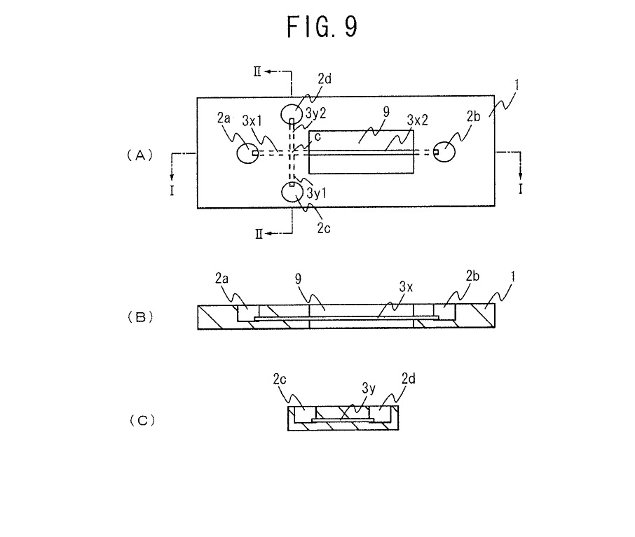

FIG. 9 shows an analysis chip of this example. In FIG. 9, the portions that are identical to those in FIG. 1 are given the same numbers and symbols. An analysis chip of this example has a single-piece substrate, and the plurality of fluid reservoirs are in communication with each other via capillary tubes that are members independent of the substrate. The capillary tubes are composed of four capillary tubes 3x1, 3x2, 3y1 and 3y2. One end of each of the four capillary tubes is gathered at the central portion c and connects with the others. As a result, the four capillary tubes communicate with each other internally. The substrate 1 is provided with cavities (not shown) for the insertion of four capillary tubes. The capillary tube 3x1 is inserted into the substrate 1 such that the other end thereof is located on the bottom surface of the first introduction reservoir 2a. The capillary tube 3x2 is inserted into the substrate 1 such that the other end thereof is located on the bottom surface of the first recovery reservoir 2b. The capillary tubes 3x1 and 3x2 serve as the capillary channel for sample analysis 3x. The capillary tube 3y1 is inserted into the substrate 1 such that the other end thereof is located on the bottom surface of the second introduction reservoir 2c. The capillary tube 3y2 is inserted into the substrate 1 such that the other end thereof is located on the bottom surface of the second recovery reservoir 2d. The capillary tubes 3y1 and 3y2 serve as the capillary channel for sample introduction 3y. The plurality of fluid reservoirs 2a to 2d are each formed as a concave portion in the substrate 1. The substrate 1 has a rectangular parallelepipedic opening (window) 9 on the first recovery reservoir 2b side relative to the capillary channel for sample introduction 3y. Otherwise, an analysis chip of this example is of the same configuration as the analysis chip shown in FIG. 1.

An analysis chip of this example can be produced, for example, as follows. However, an analysis chip may be produced by methods other than the production method described below.

For example, a substrate that is formed from the same material as the lower substrate 1 of the analysis chip shown in FIG. 1 can be used as the substrate 1.

In an analysis chip of this example, the length, the width and the thickness of the substrate 1 correspond to the maximum length, the maximum width and the maximum thickness of the whole chip, as described above. Therefore, the length, the width and the thickness of the substrate 1 are arranged to be identical to the maximum length, the maximum width and the thickness of the whole chip as described above.

The inner diameter of each of the four capillary tubes is the same as the maximum diameter of the capillary channel described above. The length of each of the four capillary tubes is determined according to the maximum length of the capillary channel for sample analysis 3x and the maximum length of the capillary channel for sample introduction 3y.

An example of a process for producing an analysis chip of this example is described below. However, an analysis chip may be produced by processes other than the production process described below.

First, the substrate 1 is prepared. A method for forming the four fluid reservoirs 2a to 2d and the opening (window) 9 in the substrate 1 is not particularly limited and, for example, the fluid reservoirs can be formed by the same method used for forming the four fluid reservoirs 2a to 2d of the analysis chip shown in FIG. 6. The fluid reservoirs 2a to 2d and the opening (window) 9 may each be formed separately or may all be formed simultaneously. When the four fluid reservoirs 2a to 2d and the opening (window) 9 are formed separately, they may be formed in any order. Forming all four fluid reservoirs 2a to 2d and the opening (window) 9 simultaneously by an aforementioned method that uses a metal mold or a like method requires a small number of steps and is thus preferable.

Next, the four capillary tubes are inserted into the substrate 1. In this manner, an analysis chip of this example can be obtained.

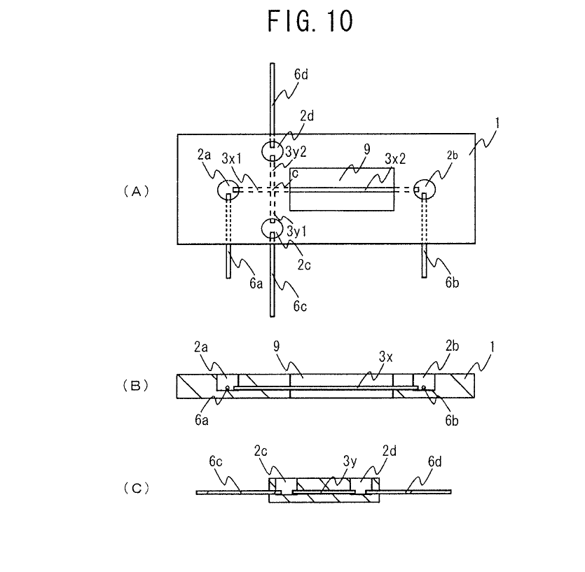

FIG. 10 shows an analysis chip of this example in which a plurality of electrodes for use with a capillary electrophoresis method are provided. In FIG. 10, the portions that are identical to those in FIG. 4 are given the same numbers and symbols. As shown in FIG. 10, in this analysis chip, the four electrodes for use with a capillary electrophoresis method 6a to 6d are embedded in the substrate 1. Otherwise, an analysis chip of this example is of the same configuration as the analysis chip shown in FIG. 4. The four electrodes 6a to 6d can be readily disposed into position by creating, in advance, introduction holes for receiving the four electrodes 6a to 6d in side surfaces of the substrate 1 when producing the upper substrate 1.

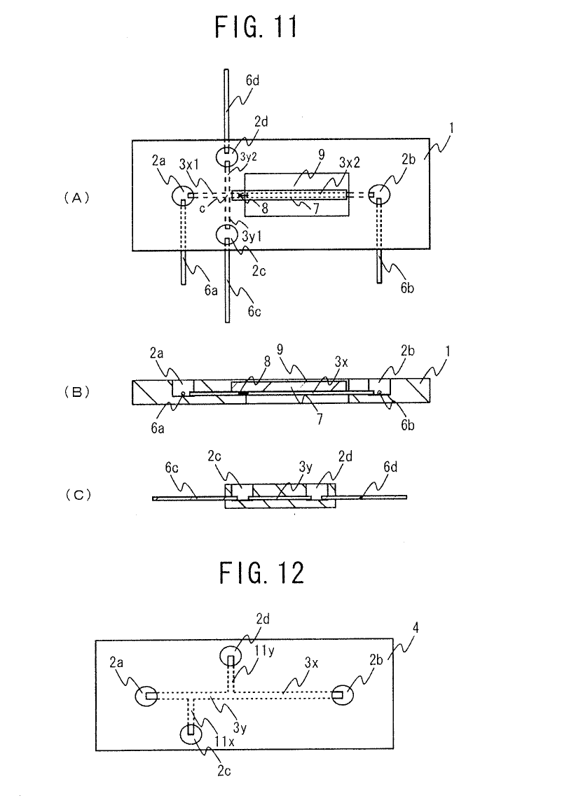

FIG. 11 shows an example of an analysis apparatus that includes an analysis chip of this example. In FIG. 11, the portions that are identical to those in FIG. 5 are given the same numbers and symbols. As shown in FIG. 11, an analysis unit (line detector) 7 is directly disposed on an aforementioned capillary tube in this analysis apparatus. Moreover, in this analysis apparatus, the substrate 1 is provided with, in addition to the cavities into which the four capillary tubes are to be inserted, a cavity into which the analysis unit (line detector) 7 is to be inserted (not shown). Otherwise, an analysis apparatus of this example has the same configuration as the analysis apparatus shown in FIG. 5. An analysis apparatus of this example is not limited by the configuration shown in FIG. 11 and, for example, a detector as in the analysis apparatus of FIG. 6 may be included. An analysis of glycosylated hemoglobin and glucose using an analysis apparatus of this example can be carried out also in the same manner as with the case where the analysis apparatus shown in FIG. 5 or FIG. 6 is used.

### Example 5

FIG. 12 shows an analysis chip of this example. In FIG. 12, the portions that are identical to those in FIG. 1 are given the same numbers and symbols. FIG. 12 is a plan view of an analysis chip of this example. As shown in FIG. 12, in this analysis chip, a groove having a shape of two “T”s combined is formed in place of a cross-shaped groove in the lower substrate 1 (not shown) and, thereby, a capillary channel for sample analysis 3x and a capillary channel for sample introduction 3y are formed. That is, first, the capillary channel for sample analysis 3x is linear, and the first introduction reservoir 2a and the first recovery reservoir 2b are in communication with each other via the capillary channel for sample analysis 3x. A first branching channel 11x branches off from a part of the capillary channel for sample analysis 3x. The first branching channel 11x is in communication with the second introduction reservoir 2c. A second branching channel 11y branches off from a part of the capillary channel for sample analysis 3x that is located on the downstream side (right-hand side on FIG. 12) relative to the first branching channel 11x. The second branching channel 11y is in communication with the second recovery reservoir 2d. The capillary channel for sample introduction 3y is formed by the first branching channel 11x, the second branching channel 11y and the part of the capillary channel for sample analysis 3x that connects the branching channels. The first branching channel 11x and the second branching channel 11y are substantially perpendicular to the capillary channel for sample analysis 3x and form together with the capillary channel for sample analysis 3x a groove having a shape of two “T”s combined. Otherwise, an analysis chip of this example is of the same configuration as the analysis chip shown in FIG. 1.

The configuration of an analysis chip of this example is not limited to the configuration shown in FIG. 12. For example, an analysis chip may be composed of a single-piece substrate as shown in FIG. 8. Moreover, an analysis chip may be provided with a plurality of electrodes for use with a capillary electrophoresis method as shown in FIG. 4 and FIG. 10 and may be suitably provided with a pretreatment reservoir as described above. A method for producing an analysis chip of this example is also not particularly limited, and may be identical to, for example, the production methods described in Examples 1 to 4 above. The configuration of an analysis apparatus that uses an analysis chip of this example is also not particularly limited and, for example, a detector as in the analysis apparatus of FIG. 5, FIG. 6, or FIG. 11 may be provided therein. Moreover, a method for analyzing glycosylated hemoglobin and glucose using the analysis apparatus is also not particularly limited, and can be carried out, for example, in the same manner as with the case where the analysis apparatus shown in FIG. 5, FIG. 6, or FIG. 11 is used.

### Example 6

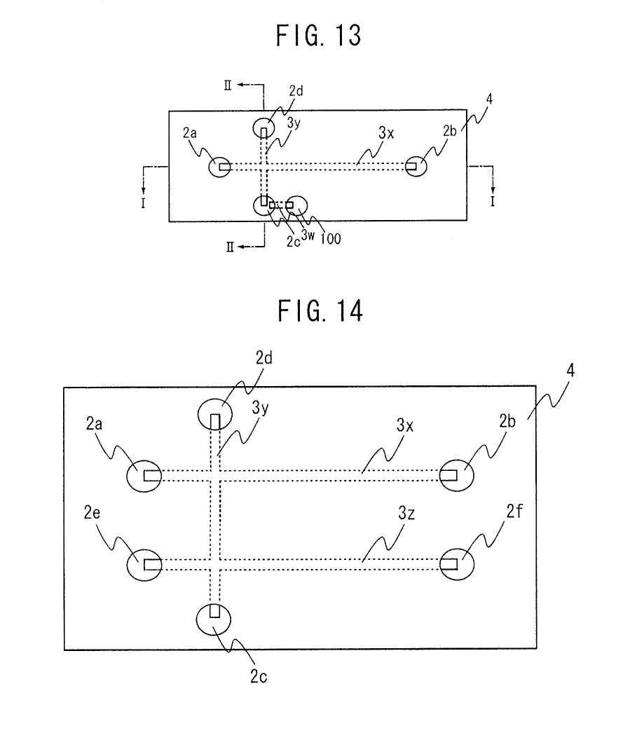

FIG. 13 shows an analysis chip of this example. In FIG. 13, the portions that are identical to those in FIG. 1 are given the same numbers and symbols. FIG. 13 is a plan view of an analysis chip of this example. In an analysis chip of this example, glucose is analyzed using a reagent as described above that develops a color in association with a redox reaction. As shown in FIG. 13, this analysis chip has a reagent reservoir 100 that is formed near the second introduction reservoir 2c. The reagent reservoir 100 is formed by sealing the bottom part of a through-hole formed in the upper substrate 4 with the lower substrate 1. The reagent reservoir 100 is in communication with the second introduction reservoir 2c via a channel 3w that is independent of the capillary channel for sample analysis 3x and the capillary channel for sample introduction 3y. The reagent reservoir 100 contains a reagent that develops a color in association with a redox reaction. The reagent reservoir 100 may include, for example, electrodes for use with a capillary electrophoresis method and the like. Otherwise, an analysis chip of this example is of the same configuration as the analysis chip shown in FIG. 1.

The configuration of an analysis chip of this example is not limited to that shown in FIG. 13. For example, an analysis chip may be composed of a single-piece substrate as in FIG. 9. Moreover, an analysis chip may be provided with a plurality of electrodes for use with a capillary electrophoresis method as in FIG. 4 and FIG. 10 and may be suitably provided with a pretreatment reservoir as described above. A method for producing an analysis chip of this example is also not particularly limited, and may be identical to, for example, the production methods described in Examples 1 to 4 above. The configuration of an analysis apparatus that uses an analysis chip of this example is also not particularly limited and, for example, a detector as in the analysis apparatus of FIG. 5, FIG. 6, or FIG. 11 may be included.

Furthermore, a method for analyzing glycosylated hemoglobin and glucose using the analysis apparatus is also not particularly limited, and is carried out, for example, as follows. That is, first, a sample is introduced into the second introduction reservoir 2c in the same manner as in the case where the analysis apparatus of FIG. 5, FIG. 6, or FIG. 11 is used. When there is a pretreatment reservoir as described above, the sample may be introduced thereinto. Then, the sample is moved into the reagent reservoir 100, and the glucose is analyzed there. A method for moving the sample into the reagent reservoir 100 is not particularly limited and, for example, the sample may be moved by applying a voltage to the electrodes for use with a capillary electrophoresis method provided in the reagent reservoir 100. The method for analyzing glucose is not particularly limited and, for example, an analysis can be carried out in the same manner as in the case where the analysis apparatus shown in FIG. 5, FIG. 6, or FIG. 11 is used. Thereafter, a potential difference between both ends of the capillary channel for sample introduction 3y is created in the same manner as in the case where the analysis apparatus of FIG. 5, FIG. 6, or FIG. 11 is used, and it is thus possible to analyze glycosylated hemoglobin.

### Example 7

FIG. 14 shows an analysis chip of this example. In an analysis chip of this example, the glucose is analyzed by a capillary electrophoresis method. In FIG. 14, the portions that are identical to those in FIG. 1 are given the same numbers and symbols. FIG. 14 is a plan view of an analysis chip of this example. As shown in FIG. 14, this analysis chip further includes a third introduction reservoir 2e and a third recovery reservoir 2f, and these reservoirs are in communication with each other via a capillary channel for glucose analysis 3z. The third introduction reservoir 2e and the third recovery reservoir 2f are, as with the other four fluid reservoirs, formed by sealing the bottom parts of through-holes formed in the upper substrate 4 with the lower substrate 1. The capillary channel for glucose analysis 3z, as with the other two capillary channels, is formed by sealing the upper part of a groove formed in the lower substrate 1 with the upper substrate 4. The capillary channel for glucose analysis 3z is disposed parallel to the capillary channel for sample analysis 3x, intersects with the capillary channel for sample introduction 3y, and is in communication with the capillary channel for sample introduction 3y at the intersection. Moreover, the capillary channel for glucose analysis 3z is formed nearer the introduction reservoir 2c in relation to the capillary channel for sample analysis 3x. Otherwise, an analysis chip of this example is of the same configuration as the analysis chip shown in FIG. 1.

The configuration of an analysis chip of this example is not limited to that shown in FIG. 14. For example, an analysis chip may be composed of a single-piece substrate as in FIG. 9. Moreover, an analysis chip may be provided with a plurality of electrodes for use with a capillary electrophoresis method as in FIG. 4 and FIG. 10 and may be suitably provided with a pretreatment reservoir as described above. Furthermore, for example, the capillary channel for glucose analysis 3z and the capillary channel for sample analysis 3x may be disposed inversely. That is, the capillary channel for glucose analysis 3z may be formed nearer the second recovery reservoir 2d in relation to the capillary channel for sample analysis 3x. A method for producing an analysis chip of this example is also not particularly limited, and may be identical to, for example, the production methods described in Examples 1 to 4 above.

The configuration of an analysis apparatus that uses an analysis chip of this example is also not particularly limited. For example, the third introduction reservoir 2e and the third recovery reservoir 2f may include electrodes for use with a capillary electrophoresis method (not shown) as with the other four fluid reservoirs. Moreover, the capillary channel for glucose analysis 3z may include a suitable glucose detector. The glucose detector is not particularly limited, and it may be, for example, a detector that analyzes glucose by indirect absorption spectroscopy (indirect UV detection method) or a like detector. The structure thereof is also not particularly limited, and the detector may be identical to the analysis unit 7 in the analysis apparatus of FIG. 5, FIG. 6, or FIG. 11. Otherwise, the configuration of an analysis apparatus of this example may be identical to that of the analysis apparatus of FIG. 5, FIG. 6, or FIG. 11. A method for analyzing glycosylated hemoglobin and glucose using this analysis apparatus is also not particularly limited. It may be identical to an analysis method using the analysis apparatus of FIG. 5, FIG. 6, or FIG. 11 except that a voltage is applied to both ends of the capillary channel for glucose analysis 3z and the glucose is analyzed by the glucose detector.

According to the present invention, an accurate blood sugar status can be obtained by, for example, analyzing glycosylated hemoglobin and glucose with high accuracy. It is thus possible to carry out a specific diabetic treatment for the purpose of preventing diabetic complications. Moreover, an analysis chip and an analysis apparatus of the present invention can be introduced into small-scale hospitals and the like due to the small size and the low cost of the apparatus. An analysis chip and an analysis apparatus of the present invention has a simple configuration and permits analysis to be carried out conveniently. For example, by making the analysis chip a disposable device, post-processing is eliminated, and the operation is thus more convenient. Furthermore, due to the fact that the apparatus is small and the analysis time is short, it is possible, for example, to provide an immediate diagnosis (the result of an analysis) in front of a patient.

### INDUSTRIAL APPLICABILITY

An analysis chip of the present invention enables an apparatus to be small, analysis to be simple, analysis time to be short, and analysis of glycosylated hemoglobin and glucose to be highly accurate. An analysis chip of the present invention is applicable to all technical fields where glycosylated hemoglobin and glucose are analyzed, such as laboratory tests, biochemical examinations and medical research. The intended use of the analysis chip is not limited and it is applicable to a broad range of technical fields.

1. A method of analyzing glycosylated hemoglobin and glucose using an analysis chip, wherein the glycosylated hemoglobin and glucose are analyzed by a capillary electrophoresis method, the analysis chip comprising:
a substrate, a plurality of fluid reservoirs and a capillary channel for the capillary electrophoresis method, a glycosylated hemoglobin analysis reagent, and a glucose analysis reagent,
the plurality of fluid reservoirs comprising a first introduction reservoir and a first recovery reservoir,
the capillary channel comprising a capillary channel for sample analysis,
the first introduction reservoir and the first recovery reservoir being formed in the substrate, and
the first introduction reservoir and the first recovery reservoir being in communication with each other via the capillary channel for sample analysis,
the glucose analysis reagent being a reagent that is different from the glycosylated hemoglobin analysis reagent, and
the glucose being analyzed according to a capillary electrophoresis method using the glucose analysis reagent in the capillary channel for sample analysis.

2. The method according to claim 1, wherein in the analysis chip
the plurality of fluid reservoirs further comprises a second introduction reservoir and a second recovery reservoir,
the capillary channel further comprises a capillary channel for sample introduction,
the second introduction reservoir and the second recovery reservoir are formed in the substrate,
the second introduction reservoir and the second recovery reservoir are in communication with each other via the capillary channel for sample introduction,
the capillary channel for sample analysis and the capillary channel for sample introduction intersect, and
the capillary channel for sample analysis and the capillary channel for sample introduction are in communication with each other at the intersection.

3. The method according to claim 2, wherein in the analysis chip
a first branching channel branches off from a part of the capillary channel for sample analysis,
the first branching channel is in communication with the second introduction reservoir,
a second branching channel branches off from a part of the capillary channel for sample analysis that is located on the downstream side relative to the first branching channel,
the second branching channel is in communication with the second recovery reservoir, and
the capillary channel for sample introduction is formed by the first branching channel, the second branching channel, and a part of the capillary channel for sample analysis that connects the branching channels.

4. The method according to claim 1, wherein the analysis chip has a maximum length of the whole chip in a range of 10 to 100 mm, a maximum width of the whole chip in a range of 10 to 60 mm, and a maximum thickness of the whole chip in a range of 0.3 to 5 mm.

5. The method according to claim 1, wherein, in analyzing glycosylated hemoglobin and glucose, a diluted sample prepared by diluting a sample containing a glycosylated hemoglobin and glucose with an electrophoresis running buffer is introduced into at least one reservoir among the plurality of fluid reservoirs, and a volume ratio of the sample: the electrophoresis running buffer is 1:4 to 1:99.

6. The method according to claim 1, wherein the capillary channel is filled with an electrophoresis running buffer.

7. The method according to claim 1, wherein the capillary channel has a maximum diameter in a range of 10 to 200 μm and a maximum length of 0.5 to 15 cm.

8. The method analysis chip according to claim 1, wherein in the analysis chip
further comprising a pretreatment reservoir for hemolyzing and diluting a sample containing glycosylated hemoglobin and glucose, the pretreatment reservoir and at least one reservoir among the plurality of fluid reservoirs being in communication with each other.

9. The method according to claim 8,
the glucose analysis reagent being contained in at least one reservoir among the plurality of fluid reservoirs and the pretreatment reservoir.

10. The method according to claim 1, wherein in the analysis chip
further comprising a reagent reservoir,
the reagent reservoir containing a glucose analysis reagent and being in communication with at least one reservoir among the plurality of reservoirs and the pretreatment reservoir.

11.-14. (canceled)

15. The method according to claim 1, wherein the analysis chip further comprises a detector that analyzes glucose according to an indirect UV detection method.

16. The method according to claim 1, wherein an ionic functional group is introduced into glucose to produce a glucose derivative.

17. The method according to claim 1, wherein the glycosylated hemoglobin is HbA1c.

18. The method according to claim 1, wherein in the analysis chip
the substrate comprises an upper substrate and a lower substrate,
a plurality of through-holes are formed in the upper substrate,
a groove is formed in the lower substrate,
the upper substrate is laminated onto the lower substrate,
spaces created by sealing the bottom parts of the plurality of through-holes formed in the upper substrate with the lower substrate serve as the plurality of fluid reservoirs, and
a space created by sealing the upper part of the groove formed in the lower substrate with the upper substrate serves as the capillary channel.

19. The analysis chip method according to claim 1, wherein in the analysis chip.
a plurality of concave portions and a groove are formed in the substrate,
a surface of the substrate is sealed with a sealing material that has openings at places corresponding to the plurality of concave portions,
the plurality of concave portions formed in the substrate serve as the plurality of fluid reservoirs, and
a space created by sealing the upper part of the groove formed in the substrate with the sealing material serves as the capillary channel.

20. The method according to claim 1, wherein
the analysis chip further includes a sealing material, and wherein in the analysis chip
a plurality of through-holes are formed in the substrate,
a groove is formed in the bottom surface of the substrate,
the bottom surface of the substrate is sealed with the sealing material,
spaces created by sealing the bottom parts of the plurality of through-holes formed in the substrate with the sealing material serve as the plurality of fluid reservoirs, and
a space created by sealing the lower part of the groove formed in the bottom surface of the substrate with the sealing material serves as the capillary channel.

21. The method according to claim 1, wherein in the analysis chip
the plurality of fluid reservoirs are in communication with each other via a capillary tube that is a member independent of the substrate, and the capillary tube serves as the capillary channel.

22. The method according to claim 1, wherein in the analysis chip
the plurality of fluid reservoirs each has a volume in a range of 1 to 1000 mm³.

23. The method according to claim 1, wherein
the analysis chip further comprises a plurality of electrodes for use with a capillary electrophoresis method, and
the plurality of electrodes for use with a capillary electrophoresis method are disposed such that their one ends are disposed respectably in the plurality of fluid reservoirs.

24.-25. (canceled)
